(19) United States
(12) Patent Application Publication
(10) Pub. No.: US 2015/0328139 A1
(43) Pub. Date: Nov. 19, 2015

(54) Orally Ingested or Topically Applied Skin Health and Nutrition Product

(71) Applicant: Keith Marz, Sherman Oaks, CA (US)

(72) Inventor: Keith Marz, Sherman Oaks, CA (US)

(21) Appl. No.: 14/278,462

(22) Filed: May 15, 2014

(51) Int. Cl. A61K 8/97; A61K 31/047; A61K 36/61; A23L 1/22; A61K 8/49; A61K 31/355; A61K 36/02; A61Q 19/00; A61K 8/34; A61K 36/889

(52) U.S. Cl. CPC A61K 8/975; A61K 8/345; A61K 31/047; A61K 8/97; A61K 36/61; A61K 36/889; A61K 8/498; A61K 31/355; A61K 36/02; A61Q 19/00; A23L 1/22; A61K 2800/591; A23V 2002/00

(57) ABSTRACT

A skin health and nutrition product that is comprised of a formulation of ingredients that can be orally ingested or topically applied. When orally ingested, or when topically applied, the product can aid in improving the health and appearance of a person's skin and provide general nutrition benefits. The product is designed to improve the quality of a person's skin from both oral ingestion and topical applications.

### TECHNICAL FIELD

The invention generally pertains to health enhancing products, and more particularly to a skin health and nutrition product that can be orally ingested and/or topically applied.

### BACKGROUND ART

Throughout history and in all parts of the ancient and modern world there have been “healers” endowed with the power to prevent and/or cure a variety of bodily ailments. Typically, for each specific internal ailment or each specific external ailment, a single ingredient or a mixture of ingredients are prescribed to treat the single specific ailment.

In today's medical technologies there are a myriad of internally taken medicants such as pills and tonics that are prescribed to treat a specific internal or external ailment. For each specific ailment there is prescribed a specific formulation. In particular, there are many products that claim to improve the quality and appearance of a person's skin. The mixing of the prescribed formulations is typically not recommended.

The instant invention provides a single skin health and nutrition product that can be either orally ingested or applied topically. Either oral ingestion or topical application will provide a person with all of the benefits of the product, which are especially directed at the quality and appearance of the skin.

A search of the prior art did not disclose any literature or patents that read directly on the claims of the instant invention.

### DISCLOSURE OF THE INVENTION

The orally ingested and/or topically applied skin health and nutrition product is comprised of a formulation that can improve skin health and appearance and for general nutrition. The product is designed to provide a nutrition improvement which leads to better external skin quality. The product improves the quality of a person's skin by both oral ingestion and/or topical application. In its basic configuration product is comprised of
a) a formulation of ingredients,
b) means for orally ingesting the formulation of ingredients, and
c) means for topically applying the formulation of ingredients.

Depending on the desired benefits and the preference/requirements of the user, the formulation includes essential ingredients and one optional flavor ingredient that can be added to improve the taste of the product when the product is orally ingested. The formulated ingredients are comprised of:
a) purified water which is derived by reverse osmosis, carbon filtration, ultraviolet oxidation or electrodialysis to remove impurities. The water provides the means by which the product can be produced as a homogenized solution,
b) glycerin or glycerol which is derived from animal or vegetable oils and fats is used as a solvent that functions as a catalyst that aids in mixing the formulated ingredients,
c) sorbitol which is derived from the berries of the mountain ash and is used as a softener and moisture conditioner,
d) camellia sinensis extract which is derived from a green tea extract and is used to prevent and treat a variety of cancers, improve mental alertness, lower cholesterol levels, and protect the skin from sun damage,
e) panax ginseng extract which is derived from the roots of the ginseng plant and promotes adaptogenic functions, resistance to stress and increases energy levels,
f) punica gram tum extract which is derived from a pomegranate extract and is an anti-oxidant that is rich in phytochemical compounds, flavoroids and polyphenols. Pomegranate extract provides protection against heart disease, skin cancer, and benefits the heart and blood vessels,
g) acai berry extract,
h) aloe vera extract,
i) seaweed (from multiple sources),
j) stevia which is derived from herbs and shrubs found in the sunflower family and serves as a natural alternative to sugar and artificial sweeteners.
k) potassium sorbate which is derived from the potassium salt of sorbic acid and is used as a preservative and inhibits the growth of mold and microbic and increases the shelf life of the product,
l) retinyl palmitate which equates to vitamin A and is an anti-oxidant nutrient found in foods such as eggs and fish, prevents and or reverses cancerous changes in cells in some parts of the body including the skin, and can repair skin damage caused by the sun,
m) ascorbic acid which equates to vitamin C and is a nutrient that aids in the production of collagen and functions as an anti oxidant which aids in repairing aged and photo-damaged skin,
n) alpha tocopherol which equates to vitamin E and is effective as an antioxidant particularly against toxins such as air pollution, for premenstrual syndrome, eye disorders, neurological disorders, and diabetes, and
o) flavor which is derived from a variety of fruits, and is used to improve the taste of the ingested product when orally ingested.

In view of the above disclosure, the primary object of the invention is to provide a skin health and nutrition product that can be orally ingested and/or topically applied to aid in improving the health and appearance of a person's skin and as a nutritional benefit.

In addition to the primary object of the invention it is also an object of the invention to provide a skin health and nutrition product that:
is safe to use,
is non-toxic,
is easily formulated,
can include a flavor enhancing ingredient,
is easy to apply or ingest,
can be used by adults and children,
does not require professional medical supervision,
can be produced as a liquid, a paste, a gel, a spray or a tablet, and
is cost effective from both a manufacturer's and consumer's point of view.

These and other objects and advantages of the present invention will become apparent from the subsequent detailed description of the preferred embodiment and the appended claims taken in conjunction with the accompanying drawings.

### BRIEF DESCRIPTION OF THE DRAWING

FIG. 1 is a block diagram of the orally ingested and/or topically applied skin health and nutrition product showing the flow path of the formulation of ingredients through an ingredient application means and on to orally ingested internal or topically applied external usage.

### BEST MODE FOR CARRYING OUT THE INVENTION

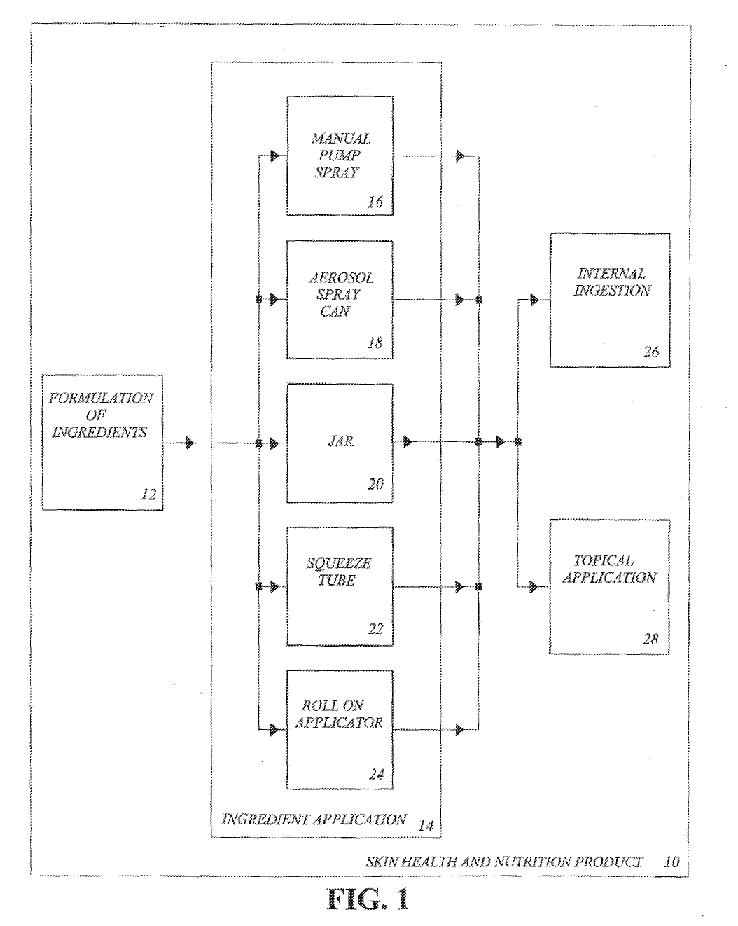

The best mode for carrying out the invention is presented in terms that disclose a preferred embodiment of a skin health and nutrition product that can be orally ingested and/or topically applied to improve the health and appearance of a person's skin and for general nutrition. The preferred embodiment of the orally ingested and/or topically applied skin health and nutrition product 10, as shown in FIG. 1, is comprised of two major elements: a formulation of ingredients 12, and an ingredient application means 14. The basic premise of the product 10 is that an identical formulation of ingredients 12 can be utilized for orally ingested internal usage 26 and/or topically applied external usage 28.

The formulation of ingredients 12 is comprised of the following percentage of ingredients:

| Ingredient | % by Weight |
|---|---|
| a) purified water | 55.38 |
| b) glycerin, | 25.00 |
| c) sorbitol, | 15.00 |
| d) camellia sinensis extract, | 0.40 |
| e) panax ginseng extract, | 0.40 |
| f) punica grana tum extract, | 0.40 |
| g) coenzyme Q10, | 0.60 |
| h) acai berry extract, | 0.40 |
| i) aloe vera extract, | 0.70 |
| j) seaweed (from multiple sources) | 0.60 |
| k) stevia, | 0.26 |
| l) potassium sorbate, | 0.25 |
| m) retinyl palmitate, | 0.18 |
| n) ascorbic acid, | 0.18 |
| o) alpha tocopherol, and | 0.15 |
| p) flavor | 0.10 |

All the formulated ingredients are considered to be non-toxic whether they are taken severally or in combination. Also, the formulation can be utilized without the flavor ingredient. If the flavor ingredient is included in the ingested formulation it can be selected from the group of flavors consisting of orange, strawberry, grape, banana or another flavor. Additionally, at least two of the flavors can be combined to produce a unique combination flavor.

The formulation of ingredients can be derived from various sources: the purified water is derived by a physical process such as reverse osmosis, glycerin is derived from animal or vegetable oils, sorbitol is derived from the berries of the mountain ash, camellia sinensis is derived from a green tea extract, panax ginseng is derived from the roots of the ginseng plant, punica grana turn is derived from a pomegranate extract, and stevia is derived from herbs and shrubs in the sunflower family. Additionally, retinyl palmitate equates to vitamin A, ascorbic acid equates to vitamin C, and alpha tocopherol equates to vitamin E.

As previously stated, the product 10 can be utilized for orally ingested internal usage 26 or topically applied for external usage 28. While an individual can use either the oral ingestion method or the topical application method, it is also possible to use both the oral and topical methods concurrently. When the two methods are used concurrently, an individual will receive the independent benefits that each method provides. In other words, both the oral ingestion method and the topical application method will function with full respective efficacy, whether one of the methods is utilized or both methods are utilized at one time. As shown in FIG. 1, the ingredient application means 14 used for the oral ingestion are preferably comprised of a manual pump spray 16, an aerosol spray can 18 pouring from a bottle or jar (not shown), or squeezing from a tube (not shown). Likewise, for topical application 28 which provides maximum efficacy for the skin, the product application means 14 can be comprised of a manual pump spray 16, an aerosol spray can 18, pouring from a bottle, a jar 20, squeezing from a tube 22 or a roll-on applicator 24.

It should be noted that the main inventive concept is the ability to provide a single skin health and nutrition product that can be effectively used when orally ingested and/or topically applied. The ingredients in the formulation can be altered and it is expected that other formulations will also be developed that can be orally ingested and/or topically applied. For example, the instant formulation can include only:
Purified water,
Glycerin,
Sorbitol,
Punica grana tum extract,
Acai berry extract,
Alpha tocopherol, and
Seaweed (from multiple sources).

These ingredients represent a basic formulation that would provide health and nutrition benefits and especially an improvement in the quality of a person's skin. As previously stated, there can potentially be any number of other formulations that are equally useful and efficacious.

While the invention has been described in detail and pictorially shown in the accompanying drawings it is not to be limited to such details, since many changes and modifications may be made to the invention without departing form the spirit and the scope thereof. Hence, it is described to cover any and all modifications and forms which may come within the language and scope of the claims.

1. An orally ingested or topically applied skin health and nutrition product comprising:
a) ingredients,
b) means for orally ingesting said ingredients, and
c) means for topically applying said ingredients.

2. The orally ingested or topically applied skin health and nutrition product as specified in claim 1 wherein said ingredients are comprised of a formulation of ingredients.

3. The orally ingested or topically applied skin health and nutrition product as specified in claim 1 wherein said formulation of ingredients comprises:

| Ingredient | % by Weight |
|---|---|
| a) purified water | 55.38 |
| b) glycerin, | 25.00 |
| c) sorbitol, | 15.00 |
| d) camellia sinensis extract, | 0.40 |
| e) panax ginseng extract, | 0.40 |
| f) punica grana tum extract, | 0.40 |
| g) coenzyme Q10, | 0.60 |
| h) acai berry extract, | 0.40 |
| i) aloe vera extract, | 0.70 |
| j) seaweed (from multiple sources) | 0.60 |
| k) stevia, | 0.26 |
| l) potassium sorbate, | 0.25 |
| m) retinyl palmitate, | 0.18 |
| n) ascorbic acid, | 0.18 |
| o) alpha tocopherol, and | 0.15 |
| p) flavor | 0.10 |

4. The orally ingested or topically applied skin health and nutrition product as specified in claim 1 wherein all the ingredients whether individually or in combination are non-toxic.

5. The orally ingested or topically applied skin health and nutrition product as specified in claim 1 wherein said ingredients are in a form selected from the group consisting of a fluid, a solid, and a powder.

6. The orally ingested or topically applied skin health and nutrition product as specified in claim 1 wherein said glycerin, which is also known as glycerol, is derived from animal and vegetable oils and fats.

7. The orally ingested or topically applied skin health and nutrition product as specified in claim 1 wherein said sorbitol is derived from the berries of mountain ash.

8. The orally ingested or topically applied skin health and nutrition product as specified in claim 3 wherein said camellia sinensis extract is derived from green tea extract.

9. The orally ingested or topically applied skin health and nutrition product as specified in claim 3 wherein said panax ginseng extract is derived from ginseng extract.

10. The orally ingested or topically applied skin health and nutrition product as specified in claim 3 wherein said punica grana tum extract is derived from pomegranate extract.

11. The orally ingested or topically applied skin health and nutrition product as specified in claim 3 wherein said retinyl palmitate equates to vitamin A.

12. The orally ingested or topically applied skin health and nutrition product as specified in claim 3 wherein said ascorbic acid equates to vitamin C.

13. The orally ingested or topically applied skin health and nutrition product as specified in claim 3 wherein said to vitamin E.

14. The orally ingested or topically applied skin health and nutrition product as specified in claim 1 wherein said flavor is selected from the group of flavors consisting of orange, strawberry, grape and banana.

15. The orally ingested or topically applied skin health and nutrition product as specified in claim 1 wherein said means for orally ingesting said health product comprises a manual pump spray, an aerosol spray can, pouring from a bottle or jar, or squeezing from a tube.

16. The orally ingested or topically applied skin health and nutrition product as specified in claim 1 wherein said means for topically applying said health product is selected from the group consisting of a manual pump spray, an aerosol spray can, pouring from a bottle or jar, squeezing from a tube or a roll-on applicator.

17. The orally ingested or topically applied skin health and nutrition product as specified in claim 1 wherein said oral ingestion and said topical application are performed concurrently.

18. An orally ingested or topically applied skin health and nutrition product comprising:
a) purified water,
b) glycerin,
c) sorbitol,
d) punica grana tum extract,
e) acai berry extract,
f) alpha tocopherol,
g) seaweed,
h) means for orally ingesting said formulation of ingredients, and
i) means for topically appling said formulation of ingredients.

19. The orally ingested or topically applied skin health and nutrition product as specified in claim 18 wherein the said formulation can be either orally ingested or topically applied.

20. The orally ingested or topically applied skin health and nutrition product as specified in claim 18 wherein said oral ingestion and said topical application are performed concurrently.
